(19) United States
(12) Patent Application Publication
(10) Pub. No.: US 2009/0255006 A1
(43) Pub. Date: Oct. 8, 2009

(54) Transgenic Zebrafish

(75) Inventors: Scott Dougan, Athens, GA (US); Xiang Fan, Watkinsville, GA (US)

Correspondence Address: Pabst Patent Group LLP, 1545 PEACHTREE STREET NE, SUITE 320, ATLANTA, GA, 30309, US

(73) Assignee: University of Georgia Research Foundation, Inc.

(21) Appl. No.: 12/410,810

(22) Filed: Mar. 25, 2009

Related U.S. Application Data
(60) Provisional application No. 61/039,613, filed on Mar. 26, 2008.

(51) Int. Cl. A01K 67/027; C12N 5/00

(52) U.S. Cl. 800/20; 435/325

(57) ABSTRACT

Transgenic zebrafish containing sqt genomic sequences driving expression of a reporter gene are provided. This line faithfully reproduces the spatiotemporal expression pattern of endogenous sqt, and at the late blastula stage is expressed in the YSL as well as in the blastomeres. The data show that expression in embryonic and extra-embryonic tissues is controlled by separable regulatory elements, including at least two elements that mediate the response to Nodal signals in different cell types. An element upstream of the transcription start site mediates the response to Nodal signaling specifically in the EVL cells. By contrast, a conserved Nodal response element (NRE) in the first introns is required for transgene expression in the blastomeres. The data show that expression of the transgene in the blastomeres depends on Nodal signaling activity. Furthermore, expression of sqt and cyc in the blastomeres depends upon Nodal signals from the YSL. These experiments suggest that Nodal signals in the YSL act to induce nodal-related gene expression in the embryo margin by activating the Nodal autoregulatory pathway. Targeted depletion of Nodal signals from the YSL results in embryos lacking endoderm and head mesoderm, similar to the defects observed in mice lacking Nodal function in the visceral endoderm. Thus, the data provides strong genetic evidence for the functional conservation between the YSL and the visceral endoderm. This suggests a common evolutionary origin for teleost and mammalian extra-embryonic tissues, despite their profound morphological differences.

### CROSS-REFERENCE TO RELATED APPLICATION

This application claims benefit of and priority to U.S. Provisional Patent Application No. 61/039,613 filed on Mar. 26, 2008 and is incorporated by reference in its entirety.

### FIELD OF THE INVENTION

The invention is generally related to transgenic zebrafish and methods of use thereof.

### BACKGROUND OF THE INVENTION

In all multicellular organisms, cells differentiate according to their relative position in the embryo generating a highly reproducible pattern of cell fates. The body plan is established at early stages by specialized groups of cells called signaling centers. In many vertebrates, extra-embryonic tissues are the first signaling centers established and act to induce the germ layers and form the major body axes (Beddington and Robertson, Cell, 96: 195-209 (1999) and Schier and Talbot, Annu. Rev. Genet (2005). In the mouse, for example, the first cell fate decision occurs during the cleavage stages and divides the embryo into embryonic and extraembryonic lineages (Rossant and Tam, Cell Dev. Biol., 15: 573-581 (2004). At later stages, signals from the extra-embryonic ectoderm and the extraembryonic visceral endoderm are required to form the proximodistal and anterio-posterior axes (Beddington and Robertson, Cell, 96: 195-209 (1999). In teleosts, the enveloping layer (EVL) is an extraembryonic epithelial covering that forms during the cleavage stages and is sloughed off the embryo at later stages (Bouvet, Cell Tissue Res., 170: 367-382 (1976), Kimmel, et al, Development, 108: 581-594 (1990). Another extra-embryonic tissue is the yolk syncytial layer (YSL), which forms at the onset of zygotic expression (mid-blastula transition; MBT) when the blastomeres juxtaposed to the yolk fuse with each other and release their contents (Kimmel, et al., Dev. Dyn., 203: 253-310 (1995). While a potential signaling role for the EVL has not been tested, the signaling properties of the YSL are well documented (Oppenheimer, Proc. Natl. Acad. Sci. U.S.A., 20: 536-538 (1934) and Solnica-Krezel, Curr. Top. Dev. Biol., 41; 1-35 (1999). In transplant experiments, signals from the yolk can induce ectopic mesoderm (Mizuno, et al., Nature, 383: 131-132 (1996). Conversely, mesoderm and endoderm fail to form when signals from the yolk are depleted by RNase injection (Chen and Kimelman, Development, 127: 4681-4689 (2000). The essential signals produced by the YSL, however, are not known. Nodal-related proteins form a conserved subclass of the TGF-βsuperfamily that act in all vertebrates to induce the mesoderm and endoderm, pattern all three germ layers and establish the left-right body axis (Schier, Annu. Rev. Cell Dev. Biol., 19: 589-621 (2003). Consistent with these multiple functions, Nodal-related proteins are dynamically expressed throughout development. In the mouse, for example, nodal is expressed across the entire epiblast prior to gastrulation, but rapidly becomes restricted to the primitive streak and the visceral endoderm (Conlon, et al., Development 120: 1919-1928 (1994), Zhou, et al., Nature 361: 543-547. (1993). At later stages, nodal is expressed in the node and left lateral plate mesoderm (LLPM) (Collignon, et al., Nature 381: 155-158 (1996). Genetic analysis indicates that Nodal signals have different roles in each domain. Conditional mutants showed that nodal is required in the node to establish left-right asymmetry (Brennan, et al., Genes Dev., 16: 2339-2344 (2002). By contrast, the primitive streak does not form in null nodal mutants, and the resulting embryos lack all mesodermal derivatives (Conlon, et al., Development, 120: 1919-1928 (1994), Zhou, et al., Nature, 361: 543-547 (1993). Analysis of nodal mutant chimeras demonstrated that Nodal signals are required in the visceral endoderm for formation of the prechordal plate and anterior neural tissue (Varlet, et al., Development, 124: 1033-1044 (1997). Other genetic experiments indicate that Nodal signals in the epiblast act to pattern the extra-embryonic tissues (Brennan, et al., Nature, 411: 965-969 (2001). Thus in mammalian embryos, Nodal signals mediate reciprocal interactions between the embryonic and extra-embryonic tissues that are essential for embryonic development.

There are three nodal-related genes in zebrafish but only two, squint (sqt/ndr1) and Cyclops (cyc/ndr2), are required for mesoderm and endoderm formation (Feldman, et al., Nature 395: 181-185 (1998). The third nodal-related gene, southpaw (spaw/ndr3), is only expressed after gastrulation and is required to establish left-right asymmetry (Long, et al., Development, 130: 2303-2316 (2003). In the absence of sqt function, the zebrafish organizer, known as the embryonic shield, does not form (Feldman, et al., Nature, 395: 181-185 (1998). These embryos subsequently recover, however, because Cyc signals induce mesoderm and endoderm during gastrulation (Dougan, et al., Development, 130: 1837-1851 (2003), Hagos and Dougan, BMC Dev. Biol. 7: 22 (2007). At 24 hours post-fertilization (hpf), most sqt mutants are indistinguishable from wild type, but a variable minority have reduced prechordal plates and display mild cyclopia (Dougan, et al., Development, 130: 1837-1851 (2003), Heisenberg and Nusslein-Volhard, Dev. Biol., 184: 85-94 (1997). In contrast, all cyc mutants have reduced prechordal plate, resulting in cyclopia, and lack the floorplate (Hatta, et al., Nature, 350: 339-341 (1991), Rebagliati, et al., Proc. Natl. Acad. Sci. U.S.A., 95, 9932-9937 (1998b); Sampath et al., Nature, 395: 185-189 (1998). The defects in sqt;cyc double mutants are much more severe than either single mutant. These embryos lack all derivatives of the mesoderm and endoderm in the head and trunk, including the notochord, prechordal plate, trunk somites, pronephros, heart, blood and gut (Feldman et al., Nature, 395: 181-185 (1998). Thus, sqt and cyc have partially overlapping functions in germ layer formation.

Nodal signaling is mediated by a bipartite receptor complex containing the TGF-β Type I receptor, ALK4 and the Type II receptor, ActR-IIB (Reissmann et al., Genes Dev., 15: 2010-2022 (2001). In order to bind and activate the ALK4/ActR-IIB receptor complex, Nodal-related proteins require the function of the Cripto/One-Eyedpinhead (Oep) co-receptor (Cheng et al., Genes Dev., 17L: 31-36 (2003), Gritsman et al., Cell, 97: 121-132 (1999); Yeo and Whitman, Mol. Cell, 7: 949-957 (2001). ALK4 is a Ser/Thr kinase that phosphorylates cytoplasmic Smad2 and Smad3. PSmad2 or PSmad3 then dimerizes with Smad4 and the complex translocates to the nucleus, and activates transcription of target genes (Massague and Chen, Genes Dev., 14: 627-644 (2000). The Smad heterodimers associate with any of several nuclear co-factors to stimulate gene expression, the most prominent of which are the wingedhelix transcription factor FoxH1 and the paired-like homeodomain protein, Mixer (Kunwar et al., Development 130: 5589-5599 (2003). A few direct transcriptional targets of this pathway have been identified, including the nodal-related genes themselves (Meno et al., Mol. Cell 4, 287-298 (1999). Conserved elements in the introns of Xenopus xnr1 and mouse nodal mediate the autoregulatory response (Brennan et al., Nature, 411: 965-969 (2001); Hyde and Old, Development, 127: 1221-1229 (2000); Osada et al., Development, 127: 2503-2514 (2000). In both species, this element drives expression in the LLPM after gastrulation (Hyde and Old, Development, 127: 1221-1229 (2000); Osada et al., Development, 127: 2503-2514 (2000); Saijoh et al., Mol. Cell, 5: 35-47 (2000). At earlier stages, transcription factors acting on this element boost expression levels in the margin in frog embryos, and mediate expression in the epiblast of mouse embryos (Brennan et al., Nature, 411: 965-969 (2001); Hyde and Old, Development, 127: 1221-1229 (2000); Osada et al., Development, 127: 2503-2514 (2000). sqt is initially expressed during oogenesis, but its function during these stages is controversial (Gore et al., Nature, 438: 1030-1035 (2005); Gore and Sampath, Mech. Dev., 112: 153-156 (2002); Hagos et al., Dev. Biol., 7: 22 (2007); Schier, Annu. Rev. Genet., (2005). In the zygote, sqt and cyc are expressed in three independent phases (Rebagliati et al., Dev. Biol., 199: 261-272 (1998a). sqt expression initiates in dorsal blastomeres soon after MBT (3 hpf), under control of the dorsal determinant β-catenin (Bellipanni et al., Development, 133: 1299-1309 (2006); Dougan et al., Development, 130: 1837-1851 (2003). After initiation, sqt expression extends into the YSL and the EVL (Erter et al., Biol., 204: 361-372 (1998); Feldman et al., Nature, 395: 181-185 (1998). Although overexpression experiments demonstrated that Sqt signals in the YSL could induce overlying blastomeres to become dorsal mesoderm, it is not known if sqt is required in the YSL (Erter et al., Biol., 204: 361-372 (1998); Feldman et al., Nature, 395: 181-185 (1998). During the late blastula stages, sqt and cyc are co-expressed in all marginal blastomeres. Two lines of evidence indicate that expression in the marginal ring is independent of the earlier expression of sqt in the dorsal blastomeres. First, overexpressing β-catenin induces ectopic expression of sqt at 3.5 hpf, but has no effect on expression at the margin (Dougan et al., Development, 130: 1837-1851 (2003). Second, depletion of β-catenin eliminates the early dorsal expression of sqt, but does not effect sqt expression in the marginal ring (Bellipanni et al., Development, 133: 1299-1309 (2006); Kelly et al., Development, 127: 3899-3911 (2000). Although the T-box transcription factor VegT induces marginal expression of the nodal-related genes in Xenopus, the factors that induce this phase of nodalrelated gene expression in zebrafish are not known (Stennard, Curr. Bio., 8: R928-R930 (1998); White and Heasman, Mol. Dev. Evol., (2007). Expression of both sqt and cyc at this stage is maintained by an autoregulatory loop (Meno et al., Mol. Cell, 4: 287-298 (1999). In the third phase, sqt expression during gastrulation is maintained in a few blastomeres at the dorsal midline, called dorsal forerunners (Erter et al., Dev. Biol., 204: 361-372 (1998); Feldman et al., Nature, 395: 181-185 (1998); Rebagliati et al., Dev. Biol., 199: 261-272 (1998a). By contrast, cyc transcripts accumulate in the axial mesoderm (Rebagliati et al., Proc. Natl. Acad. Sci. U.S.A., 95: 9932-9937 (1998b); Sampath et al., Nature, 395: 185-189 (1998).

### SUMMARY OF THE INVENTION

Transgenic zebrafish containing sqt genomic sequences driving expression of a reporter gene are provided. This line faithfully reproduces the spatiotemporal expression pattern of endogenous sqt, and at the late blastula stage is expressed in the YSL as well as in the blastomeres. The data show that expression in embryonic and extra-embryonic tissues is controlled by separable regulatory elements, including at least two elements that mediate the response to Nodal signals in different cell types. An element upstream of the transcription start site mediates the response to Nodal signaling specifically in the EVL cells. By contrast, a conserved Nodal response element (NRE) in the first intron is required for transgene expression in the blastomeres. The data show that expression of the transgene in the blastomeres depends on Nodal signaling activity. Furthermore, expression of sqt and cyc in the blastomeres depends upon Nodal signals from the YSL. These experiments suggest that Nodal signals in the YSL act to induce nodal-related gene expression in the embryo margin by activating the Nodal autoregulatory pathway. Targeted depletion of Nodal signals from the YSL results in embryos lacking endoderm and head mesoderm, similar to the defects observed in mice lacking Nodal function in the visceral endoderm (Brennan et al., Nature, 411: 965-969 (2001); Varlet et al., Development, 124: 1033-1044 (1997). Thus, the data provides strong genetic evidence for the functional conservation between the YSL and the visceral endoderm. This suggests a common evolutionary origin for teleost and mammalian extra-embryonic tissues, despite their profound morphological differences.

Methods for harvesting embryonic endoderm cells and embryonic mesoderm cells are also provided. Briefly, the embryos of the disclosed transgenic zebrafish are dissociated and the embryonic cells are sorted, for example using a fluorescence-activated cell sorter. Endoderm and mesoderm cells fluoresce as a result of the reporter gene. The type of cell harvested can be regulated by using embryos at a specific stage of development or time of development.

### BRIEF DESCRIPTION OF THE DRAWINGS

FIG. 1A is a diagram of the sqt locus in BAC157J11. FIG. 1B sqt has three exons (rectangles) separated by two introns, 631 bp and 81 bp long, respectively. The start of transcription is indicated (arrow). Key restriction sites and the vector gene encoding chloramphenicol resistance are depicted. The GFP-FRT-neo-FRTcassette is diagrammed below the BAC, showing the site of integration in the first exon. The resulting engineered BAC clone is depicted below. After integration and excision of the gene encoding neomycin resistance, the gfp coding region replaces the sqt first exon. GFP rectangle=GFP sequences; NEO rectangle=neomycin resistance gene; red squares=FRT recombination sites; blue rectangle=sqt coding sequences. FIG. 1B is a diagram of p10sqtGFP following excision from the BAC by gap-repair. HindIII sites are indicated with “H”. Red lines indicate the sequences used to generate Tg-SqtapGFP and its derivatives. FIGS. 1C to 1E are fluorescent micrographs of a living, 5 hpf embryo injected with BAC157J11sqtGFP at the one-cell stage. Fluorescence appears throughout the margin, including the YSL, EVL and blastomeres. Perduring GFP expression is also observed in some cells farther from the margin. FIG. 1D is a low magnification fluorescent image of a living, 7 hpf embryos injected with BAC157J11 sqtGFP. Fluorescence is observed in the dorsal forerunners (white arrows), Perdurant expression is also detected in blastomeres and EVL cells farther from the margin. FIG. 1E is a fluorescent image of a living 5 hpf embryo injected with p9.9sqtGFPi at the one-cell stage. GFP fluorescence is detected around the entire margin, in the blastomeres, EVL and YSL. K=KpnI; X=XhoI; Xb=XbaI; E=EcoR1; N=Not1; H=HindIII.

FIG. 2A is a diagram of three constructs used to make stable transgenic lines with the Tol2 transposase. Each line consists of the GFP reporter gene and 1.4 kb of DNA from the sqt genomic region upstream of the transcription start site. Tg-SqtapGFPi contains 600 bp of the sqt first intron inserted downstream of the GFP poly-adenylation signal. Tg-SqtapGFP lacks the entire first intron. Tg-SqtapGFPiΔNRE is identical to Tg-SqtapGFPi, except that it lacks the 68 bp NRE sequence, shown below (SEQ ID NO:18). The FoxH1 consensus sites are highlighted in red; Smad consensus binding sites are in black bold. Photomicrographs of transgenic zebrafish embryos showing a time-course of gfp expression in Tg-SqtapGFPi (FIGS. 2B-2E), Tg-SqtapGFP (FIGS. 2F-2I) and Tg-SqtapGFPiΔNRE (FIGS. 2J-2M). In all lines, gfp mRNA is induced in dorsal blastomeres soon after MBT (2B, 2F, 2J). At 5 hpf, gfp is expressed in a ring around the entire margin in all lines (2C, 2G, 2K). The ring of expression in Tg-SqtapGFP (2G) and Tg-SqtapGFPiΔNRE (2K) is thinner than the ring in Tg-SqtapGFPi. At 6 hpf, gfp expression in Tg-SqtapGFPi (2D) and Tg-SqtapGFPiΔNRE (2L) is absent from the margin, but expression persists in Tg-SqtapGFP (arrows) (2H). At 8hpf, gfp is expressed in the dorsal forerunner cells in all three lines (red arrowheads) (2E, 2I, 2M). In Tg-SqtapGFP, expression persists in marginal EVL cells throughout gastrulation (arrows) (2I). In all three lines, ectopic gfp expression is detected at the midline (black arrowheads) (2E, 2I, 2M). (2N) The FoxH1 consensus binding sites in the sqt intron are conserved in Drosophila melanogaster (Dr) (SEQ ID NO:19), Tetraodon nigroviridis (Tn) (SEQ ID NO:20), Takifugu rubrifes (Tr) (SEQ ID NO:21), Xenopus laevis (Xl) (SEQ ID NO:22), and Mus musculus (Mm) (SEQ ID NO:23).

FIGS. 3A-H are photomicrographs of transgenic zebrafish embryos showing expression of gfp in the blastomeres. Distribution of gfp mRNA in sections of 5 hpf Tg-SqtapGFPi (3A, 3E, 3G), Tg-SqtapGFP (3B, 3F) and Tg-SqtapGFPiΔNRE (H) embryos stained to reveal gfp mRNA. Whole mounts of Tg-SqtapGFPi (3C) and Tg-SqtapGFP (3D) are also depicted. In all sections, the membrane that separates the YSL from the blastomeres is highlighted in red. In Tg-SqtapGFPi, gfp transcripts are localized in the EVL (arrow) (3A), the blastomeres, and the YSL. (3B) In Tg-SqtapGFP, gfp is expressed predominantly in the YSL and EVL (arrow). Expression is also observed in rare blastomeres adjacent to the YSL or EVL. (3C, 3E) In response to activation of the Nodal pathway by ubiquitous expression of TARAM-D, gfp is globally expressed in Tg-SqtapGFPi embryos. In section, gfp transcripts are observed in all blastomeres, EVL cells (3E, arrows), and the YSL. (3D, 3F) In Tg-SqtapGFP embryos, TARAM-D induces gfp expression throughout the embryo (3D), but only in the EVL cells (3F, arrow). (3G) When Nodal signaling is blocked by treatment with SB-505124 in Tg-SqtapGFPi embryos, gfp expression is reduced, but not eliminated in the YSL and EVL, and is eliminated in the blastomeres. (3H) In Tg-SqtapGFPiΔNRE embryos, gfp expression is restricted to the EVL (arrow) and YSL.

FIGS. 4A-D are photomicrographs showing expression of components of the Nodal-signaling pathway. Sections of wild type embryos at 5 hpf (4A) or 4.3 hpf (4B-4D) embryos stained for cyc (4A), oep (4B), bon/mixer (4C) or foxh1 mRNA (4D). (4A) In 5 hpf embryos, cyc transcripts are distributed in a punctate pattern in marginal blastomeres within 5 rows of the YSL, and are also detected in the YSL (arrows). (4B) oep is expressed in all blastomeres and EVL cells, but transcripts are excluded from the YSL. (4C) bon/mixer is expressed exclusively in the blastomeres within 3-4 rows of the YSL. bon/mixer mRNA is not detected in the YSL or EVL. (4D) foxh1 transcripts are found throughout the embryo, including all blastomeres and the YSL.

FIGS. 5A-5J are photomicrographs of zebrafish embryos. Images of live 5 hpf (5A), 6 hpf (5C, 5E, 5G, 5I), or 24 hpf (5B, 5D, 5F, 5H) embryos after targeted coinjection into the YSL of sqtMO and cycMO, or appropriate 5 bp mismatched control MOs. After co-injection of sqt and cyc mismatched MOs, the embryonic shield forms normally (5A, arrow) and the body axis is indistinguishable from wild type (5B). After co-injection of the sqtMIS MO and cycMO, the embryonic shield forms (5C, arrow) and the body axis is normal (5D). Embryonic shields do not form in many embryos following injection of the sqtMO and cycMIS MO (5G). The majority of these embryos appear normal at 24 hpf (5H), but many have reduced cardiac tissue. (5I, 5J) After co-injection of sqtMO and cycMO, the vast majority of embryos lack embryonic shields (5G). At 24 hpf, these embryos display severe cyclopia and have reduced notochord due to defects in axial mesoderm patterning (5J). Paraxial mesoderm is less affected, as somites form in embryos co-injected with sqtMO and cycMO (5J).

FIGS. 6A-6L are photomicrographs of transgenic zebrafish embryos. Embryos were injected with sqt and cyc mismatch MOs (6A, 6C, 6E, 6G, 6I, 6K) or sqt and cyc MOs (6B, 6D, 6F, 6H, 6J, 6L) and processed for in situ hybridization for mesoderm and endoderm marker genes. Control MOs do not affect expression of gsc (6A), flh (6C), mezzo (6E), sox17 (6G), sqt (6I) or cyc (6K). By contrast, gsc expression is eliminated when sqt and cyc MOs are co-injected into the YSL (6B). In FIG. 6D flh expression is reduced, but not eliminated in these embryos. These embryos also completely lack mezzo expression (6F). Sox17 expression is eliminated from the endoderm progenitors, but not from the dorsal forerunners (6H). Extremely low levels of sqt expression are apparent when Sqt and Cyc signals are depleted from the YSL (J6). Cyc expression is greatly reduced, except for a small patch of expression in the presumed dorsal blastomeres (6L). In panels 6A-6D, 6E-6F, 6I-6L, dorsal is to the right, when apparent.

FIGS. 7A and 7B are diagrams showing the conserved roles of extra-embryonic Nodal signaling in teleosts and mammals. FIG. 7A is a schematic of a blastula stage zebrafish embryo. Sqt signals (S) in the YSL (green, black arrows) induces sqt and cyc expression in the blastomeres (red). Sqt and Cyc in the YSL and/or the blastomeres induce expression in the EVL (blue). It is not clear if Nodal signals in the blastomeres induce or maintain expression in the YSL (dashed white arrows). FIG. 7B is a schematic of a 6.0-day-old mouse embryo. Nodal signals (N) in the visceral endoderm (green, black arrows) induce Nodal expression in the epiblast (red). Nodal signals in the epiblast (white arrows) maintain nodal expression in the visceral endoderm and pattern the extra-embryonic ectoderm, also known as the trophectoderm (blue). These signaling interactions in the 6.0-day-old mouse embryo were diagrammed for simplicity, but it is not clear when they occur during normal development. Black arrows indicate Nodal signaling from extra-embryonic tissues; White arrows indicate Nodal signaling from embryonic tissues. Solid lines depict interactions that have been demonstrated by experimental data; dashed lines depict possible interactions. YSL=yolk syncytial layer; EVL=enveloping layer; VE=visceral endoderm; TE=trophectoderm.

### DETAILED DESCRIPTION OF THE INVENTION

### I. Definitions

The “Tol2 transposon system” to generate transgenic zebrafish is known in the art (Kawakami, K. Genome Biol.; 8(Suppl 1): S7 (2007)). The medaka fish Tol2 element is an autonomous transposon that encodes a fully functional transposase. The transposase protein can catalyze transposition of a transposon construct that has 200 and 150 base pairs of DNA from the left and right ends of the Tol2 sequence, respectively. These sequences contain essential terminal inverted repeats and subterminal sequences. DNA inserts of fairly large sizes (as large as 11 kilobases) can be cloned between these sequences without reducing transpositional activity. The Tol2 transposon system has been shown to be active in all vertebrate cells tested thus far, including zebrafish, Xenopus, chicken, mouse, and human. Additional methods for making transgenic animals include “retroviral vectors” and “lentiviral vectors”. In general such vectors include a viral particle enclosing viral nucleic acid which has been modified to incorporate the nucleic acid of interest to be carried into the target animal cells. Such vectors may be produced from the nucleic acids of any suitable virus, including but not limited to the human immunodeficiency virus, feline immunodeficiency virus, equine infection anemia virus, Moloney murine leukemia virus, etc.

“Pseudotyped retroviral vectors” and “pseudotyped lentiviral vectors” as used herein are known. In general such vectors are those in which a viral capsid is changed, a viral capsid protein is replaced, an additional viral capsid protein is added, etc., to change, alter, or broaden the cell specificity of the virus so that it is internalized by the desired target. Pseudotyped retroviral vectors are known.

“Expression sequence” as used herein typically refers to at least one promoter, enhancer, response element, or combination thereof, including the response elements ordinarily associated with the corresponding promoter and response elements from different promoters. Promoters, enhancers and response elements may be obtained from any suitable species, including reptile, amphibian (e.g., frog), avian (e.g., chicken), and mammalian species (e.g., mouse), as well as from viruses.

“Therapeutic protein” as used herein may be any protein (including peptide, active protein fragments, and fusion proteins thereof) that has therapeutic utility in treating human or animal disease. Examples include but are not limited to insulin, glucagon-like peptide 1, antibodies, histocompatibility antigens, integrins, selectin inhibitors, growth factors, postridical hormones, nerve growth hormones, blood clotting factors, adhesion molecules, bone morphogenic proteins, lectins, trophic factors, cytokines such as TGF-beta, IL-2, IL-4, alpha-IFN, beta-IFN, gamma-IFN, TNF, IL-6, IL-8, lymphotoxin, IL-S, Migration inhibition factor, GMCSF, IL-7, IL-3, monocyte-macrophage colony stimulating factors, granulocyte colony stimulating factors, multidrug resistance proteins, other lymphokines, toxoids, erythropoietin, Factor VIII, amylin, TPA, dornase-alpha, alpha-1-antitrypsin, human growth hormones, nerve growth hormones, bone morphogenic proteins, growth differentiation factors, neuregulin, urease and toxoids, and active fragments thereof, active peptides, and fusion proteins thereof.

“Reporter protein” as used herein includes but is not limited to pigment proteins, fluorescent proteins, luminescent protein, enzymes and other detectable proteins. Specific examples include but are not limited to melanin (including eumalanin and pheomelanin), carotenoids, pteridines, cyan biocbromes, aequorin, luciferase, luciferin, blue fluorescent protein, red fluorescent protein, ds red fluorescent protein, cyan fluorescent protein, yellow fluorescent protein, and green fluorescent protein (including naturally occurring and mutant variants thereof). See, e.g., U.S. Pat. Nos. 7,034,141; 7,037,645; and 6,087,476. In some embodiments the visually detectable proteins such as the fluorescent or luminescent proteins are preferred. The foregoing is to be construed as inclusive of “enhanced” proteins, including but not limited to enhanced green fluorescent protein (eGFP), which are known in the art. A “reporter gene” is a nucleic acid encoding a reporter protein.

“Fluorescent or luminescent” as used herein to describe an animal phenotype means that at least a portion of the animal is visibly fluorescent or luminescent to the ordinary human observer under usual conditions for observing fluorescence or luminescence (e.g., dimmed or reduced light, as in night-time or a darkened room, with or without the addition of supplemental illumination of the animal with an ultra-violet or “black” light). The phenotype may be exhibited as a pattern of fluorescence or luminescence on or through the skin of the animal, overall fluorescence or luminescence on or through the skin of the animal, and/or fluorescence or luminescence of the eyes of the animal, etc.

### II. Transgenic Zebrafish

The disclosed transgenic zebrafish can be a stable transgenic zebrafish, and include zebrafish larvae, zebrafish embryos and adult zebrafish. A preferred embodiment provides transgenic zebrafish in which the expression of a reporter protein is under the control of sqt regulatory sequences. For example, transgenic animals that express a reporter protein in specific cells or tissue can be produced by introducing a nucleic acid into fertilized eggs, embryonic stem cells or the germline of the animal, wherein the nucleic acid is under the control of a specific promoter which allows expression of the nucleic acid in specific types of cells (e.g., a promoter which allows expression only in mesoderm or ectoderm). As used herein, a protein or gene is expressed predominantly in a given tissue, cell type, cell lineage or cell, when 90% or greater of the observed expression occurs in the given tissue cell type, cell lineage or cell.

Additional expression sequences used to drive expression of the reporter proteins can be isolated by one of skill in the art, for example, by screening a genomic zebrafish library for sequences upstream of the zebrafish gene of interest. The expression sequences can include a promoter, an enhancer, a silencer and necessary information processing sites, such as ribosome binding sites, RNA splice sites, polyadenylation sites and transcriptional terminator sequences.

By utilizing a transgenic zebrafish that expresses a fluorescent protein under the control of sqt sequences, the embryonic mesoderm and ectoderm can be visualized in the developing embryo and in later states of zebrafish development. Thus, the disclosed transgenic zebrafish can be used to isolated mesoderm and ectoderm embryonic tissues that fluoresce.

In certain embodiments the transgenic zebrafish includes the GFP reporter gene and 1.4 kb of DNA from the sqt genomic region upstream of the transcription start site. One embodiment of transgenic zebrafish includes the Tg-SqtapGFPi construct which contains 600 bp of the sqt first intron inserted downstream of the GFP poly-adenylation signal. Another embodiment contains the Tg-SqtapGFP construct which lacks the entire first intron. Still another embodiment contains the Tg-SqtapGFPiΔNRE construct which is identical to Tg-SqtapGFPi, except that it lacks the 68 bp NRE sequence.

### III. Nucleic Acid Constructs and Vectors

Heterologous nucleic acids of interest (e.g., those encoding a therapeutic, detectable or reporter protein) can be operatively associated with expression sequences operative in animals and inserted into suitable vectors for transfecting animal cells in accordance with known techniques, or variations thereof that will be apparent to those skilled in the art. See, e.g., U.S. Pat. Nos. 6,730,822; 6,380,458; and 5,932,780.

Retroviral and lentiviral vectors are known. See, e.g., U.S. Pat. Nos. 6,949,242; 6,838,280; 6,323,195; 6,303,116; 6,107,478; and 4,980,286. When the vectors are replication deficient, they can be produced in helper cell lines in accordance with known techniques. See, e.g., U.S. Pat. Nos. 6,712,612; 5,124,263; 4,861,719; and 4,650,764. Pseudotyped vectors are known and retroviral and lentiviral vectors can be pseudotyped in accordance with known techniques. See, e.g., U.S. Pat. Nos. 6,863,884; 6,849,454; 6,544,779; 6,479,281; 6,117,681; 5,739,018; 5,670,354; and 5,512,421; see also J. Yee et al., Methods Cell Biol., 43:99-112 (1994); J. Burns et al., Proc. Natl Acad. Sci USA, 90:833-8037 (1993).

Expression sequences including promoters, enhancers, response elements and combinations thereof (sometimes referred to as “regulatory elements”) useful for producing the disclosed zebrafish are known. See, e.g., U.S. Pat. Nos. 6,730,822; 6,380,458; and 5,932,780. The expression sequences may be constitutively active or inducible (e.g., tissue-specific). Examples include but are not limited to the CMV promoter, beta-actin promoter, the RSV promoter, crystalline promoters such as the alpha and delta crystalline promoters, the mylz2 promoter, the PGK promoter, the myosin heavy chain promoter, the myosin light chain promoter, the cardiac myosin promoter, and the keratin promoter. See, e.g., U.S. Pat. Nos. 6,949,242; 6,897,045; and 6,784,289. In some embodiments crystalline promoters and/or enhancers, such as the delta crystalline promoter and/or enhancers, are preferably included in the expression sequence.

A nucleic acid encoding the expression product (sometimes also referred to as a protein of interest) is operatively associated with the expression sequence to form what is sometimes referred to as an “expression cassette” in accordance with known techniques. If desired, insulators can be included upstream, downstream, or both upstream and downstream from the expression sequence and associated nucleic acid encoding the expression product, in accordance with known techniques. See, e.g., U.S. Pat. Nos. 6,395,549; 6,229,070; 6,100,448; and 5,610,053. Also if desired, scaffold attachment regions can be included upstream, downstream, or both upstream and downstream from the expression sequence and associated nucleic acid encoding the expression product, in accordance with known techniques. See, e.g., U.S. Pat. Nos. 6,239,328; 6,100,448; 5,773,695; and 5,773,689.

### IV. Methods for Harvesting Mesodermal and Endodermal Cells

Another embodiment provides methods and compositions for obtaining mesodermal embryonic cells and endodermal embryonic cells using the disclosed transgenic zebrafish. One embodiment provides harvesting embryos from transgenic zebrafish having reporter gene expression controlled by sqt gene regulatory elements. The cells of the embryos are dissociated using conventional techniques (Vallone, D., et al., Methods Mol Biol., 362: 429-441 (2007). If the reporter gene encodes a protein that fluoresces, the cells obtained from the embryos are exposed to electromagnetic radiation sufficient to enable the protein to fluoresce. For example, green fluorescent protein is typically excited with light having a wavelength of 395 nm. Emissions from green fluorescent protein are typically taken at its emission peak wavelength of 509 nm. Harvesting of the cells can be automated using for example, a fluorescence-activated cell sorter. The type of cell, endoderm or mesoderm, can be selected by harvesting embryos at specific stages of development and then sorting the cells obtained from the embryos. The lines label presumptive mesoderm and endoderm cells in pregastrula stage embryos, and the dorsal forerunner cells and Kuppfer's vesicle cells during gastrulation and in the somite stages. Finally, it will be appreciated that the cells can be sorted manually, if necessary.

Dissociation of pregastrula stage embryos can be accomplished by incubating them in calcium free medium. Physical dissociation is the preferred method at all stages.

Once the cells have been sorted, the cells can be used in tissue engineering to develop tissue constructs or they can be used in assays to identify modulators of mesoderm or endoderm development. One embodiment provides a population of mesodermal or endodermal embryonic zebrafish cells wherein the cells comprising a reporter gene operably linked to sqt gene regulator elements, for example the regulatory elements described in the Examples. By “operably linked” is meant that the nucleic acid sequence encoding a protein of interest, i.e., a reporter protein, and transcriptional regulatory sequences are connected in such a way as to permit expression of the nucleic acid sequence when introduced into a cell. Typically, the cell populations will be enriched for either embryonic endodermal cells or embryonic mesodermal cells. Generally greater than 80%, 85%, 90%, or 95% of the cell population is either endodermal cells or mesodermal cells. In certain embodiments the cell population contains only endodermal; cells or only mesodermal cells.

The embryonic cell populations can be packaged in a container. The container of cells can be part of a kit that includes instructions for using the cells and optionally additional reagents for culturing the cells. The cells can optionally be cryopreserved in the container using conventional techniques.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### EXAMPLES

### Materials and Methods

### Zebrafish Strain and Morpholino Injections

Wild-type embryos were obtained from natural crosses of Wik fish. Collected embryos were maintained at 28.5° C. and staged according to morphological criteria (Kimmel et al., Dev. Dyn., 203:253-310 (1995). Translation blocking morpholinos against sqt and cyc have been described previously (Feldman and Stemple, Genesis, 30: 175-177 (2001); Karlen and Rebagliati, Genesis, 30: 126-128 (2001). The MO sequences are as follows: sqtMO: 5′-ATGTCAAATCAAGGTAATAATCCAC-3′ (SEQ ID NO:1); sqtMIS: ATcTgAAAATgAAGcTAATAATgCAC-3′ (SEQ ID NO:2); cycMO: 5′-GCGACTCCGAGCGTGTGCATGATG-3′ (SEQ ID NO:3); cycMIS: 5′-GCcACTgCGAaGTGTGgTcAT-3′(SEQ ID NO:4). Nucleotides in lower case represent mismatches. The sqtMOs were tagged with lissamine, while the cycMOs were tagged with fluorescein. Embryos were injected with each MO at 3 hpf soon after formation of the YSL. The distribution of the MO in the YSL was verified 2 h after injection. Embryos with mislocalized MOs were removed from the experiments.

### BAC Recombineering

BAC 157J11 (CHORI-211 library) was identified as a 120-kb clone containing the entire sqt locus. The sqt first exon was replaced with the eGFP open reading frame by homologous recombination (Lee et al., Genomics, 73: 5645 (2001); Yu et al., Proc. Natl. Acad. Sci. U.S.A., 97: 5978-598 (2000). The pCS2EGFP-FRT-neo-FRT plasmid was generated by inserting the sacII FRT-neo-FRT cassette from pIGCN21 into pCS2EGFP (Lee et al., Genomics, 73: 56-65 (2001). The following primers were used to generate a recombination fragment: Forward targeting primer: 5′-CAGAGACTTTATTTCAATAACTGCGTGTGGATTATTACCTTGATTTGA CATGGTGAGCAAGGGCGAGGA-3′ (SEQ ID NO:5); Reverse targeting primer: 5′-ACTTTTAGCGACGAGGCTCAAGACGGAGTCAGACTCGTAAAGAGTTG GATTCTATTCCAGAAGTAGTGAG-3′ (SEQ ID NO:6).

Nucleotides in bold are homologous to sqt genomic sequences; the other nucleotides are homologous to the eGFP-FRT-neo-FRT cassette. This fragment was inserted into BAC 157J11 by homologous recombination, as described (Lee et al., Genomics, 73: 56-65 (2001). To generate p9.9sqtGFPi, the gap repair method was used to excise the eGFP open reading frame, the first intron, and 9.9 kb upstream sequences from the modified BAC 157J11 (Liu et al., Genome Res., 13: 476-484 (2003). The gap-repair targeting vector was constructed in pCS2+, and consisted of a 490-bp fragment homologous to a region 9.9 kb upstream of the gfp open reading frame and a 390-bp fragment homologous to sequences in the first intron. The upstream fragment was amplified from a preparation of BAC DNA using the following primers: Forward: 5′-TCATGGATCCGAAGATCAATTCAATCCCAT-3′ (SEQ ID NO:7); Reverse: 5′-TTACTCGAGCTAAATCAACGCTTAGACTT-3′ (SEQ ID NO:8). The forward primer contained a BamH1 site, and the reverse primer contained an XhoI site. To generate the downstream fragment, the following primers were used: Forward: 5′-TTGACCTCGCTAGACGCTGTAGCCTTGA-3′ (SEQ ID NO:9); Reverse: 5-TCCATTCTAGAAGTGTTTAGGCCAGACAGGT-3′ (SEQ ID NO: 10). The forward primer contained an Xho1 site and the reverse primer contained an XbaI site. The upstream and downstream PCR products were cut with BamH1 and XhoI, or XhoI and XbaI, respectively, and were inserted into the BamH1 and XbaI sites of pCS2+ in a single, triple-ligation reaction. The resulting plasmid, except for the XhoI site, was amplified by PCR using the following two primers: Clockwise Primer: 5′-CTAGACGCTGTAGCCTTGAT-3′ (SEQ ID NO:11); Counterclockwise primer: 5′-CTAAATCAACGCTTAGACTT (SEQ ID NO:12). The PCR product was digested by Dpn in order to minimize false positives arising from vector DNA and transformed into bacteria containing the modified BAC157J11. The resulting plasmid, p9.9sqtGFPi, includes 9.9 kb upstream of GAP and the first 603 bp of the 631 bp first intron.

Deletion Analysis of p9.9SqtGFPi

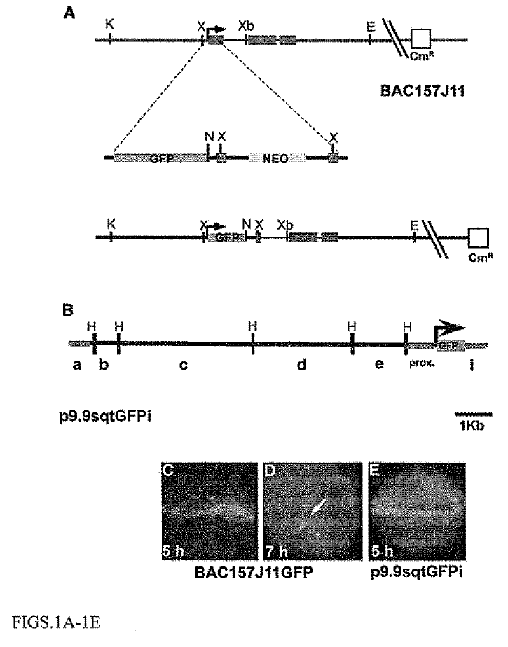

p9.9sqtGFPi was digested with HindIII, which divided the upstream region into five distal fragments and one proximal fragment, which consisted of the GFP open reading frame (G), 923 bp of genomic sequences upstream (p) and 603 bp of the first intron (i) (FIG. 1). When injected into embryos, the proximal fragment, pGi, did not express GFP protein in the margin, except for variable expression in a few random cells. The sizes of the distal HindIII fragments are as follows: fragment a=431 bp; b=746 bp; c=3669 bp; d=2629 bp; e=1456 bp. To determine which of the distal fragments contained the necessary sequences to drive reporter gene expression in the margin, each HindIII fragment was ligated to pGi in pBluescript (Stratagene, La Jolla, Calif.). Each of the five resulting constructs were injected into embryos and assayed for GFP fluorescence during the blastula stages (5 hpf), and for expression in the Kupffer's vesicle at 18 hpf. pSqtapGFPi was the only construct to consistently display strong fluorescence in the entire margin and Kupffer's vesicle. To determine if the ‘a’ fragment was capable of driving GFP expression on its own, it was ligated to the pax6 p0 promoter, which has been previously shown to work in zebrafish (Lakowski et al., Dev. Biol., 2007). Fluorescence was not observed when this construct was injected into embryos, indicating that the ‘a’ fragment is not sufficient to drive GFP expression. pSqtapGFPi was used as the starting point for further analysis. To delete the 68 bp NRE from the sqt first intron, two PCR fragments were ligated including the sequences upstream or downstream of the NRE. To amplify the upstream fragment, the Forward primer 5′-AAATCCGCTTAGTGTGTGTGTATTA-3′(SEQ ID NO: 13) was used, which has a SacII site and the Reverse primer 5′-AATTGAATTCTAAGCATAATACATGACT-3′ (SEQ ID NO:14) was used, which has an EcoRI site. The downstream fragment was amplified with the Forward primer: 5′-AATTGAATTCAGACAATAAGAATGTTCT-3′ (SEQ ID NO:15), which contains an

EcoRI site. The Reverse primer, 5′-AATAGGGCCCTGTCTAAATGTGTATTGA-3′ (SEQ ID NO: 16), has ApaI site. The fragments were digested with EcoR1 and inserted into pSqtapG to generate pSqtapGiΔNRE.

Injection of DNA and Establishment of Stable Transgenic Lines

For analysis of transiently expressed transgenes, DNA was prepared using the Qiagen BAC or plasmid purification Kit (Qiagen, Inc, Valencia Calif.). BAC DNA was diluted to 80 ng/μl and 150 pg was injected into the blastomeres of one-cell stage embryos. BAC DNA was stored at 4° C. for up to 4 weeks. Transgene expression was observed in living embryos at the blastula stages (5 hpf). Perdurant GFP expression in the Kupffer's vesicle was also examined. The pattern, fluorescence intensity, and frequency of expression were all evaluated.

To generate stable transgenic lines, DNA fragments of interest were cloned into the BamH1 and ApaI sites of a modified Tol2 vector (Kawakami et al., Dev. Cell, 7: 133-144 (2004); Urasaki et al., Dev. Cell, 7: 133-144 (2006). 150 pg of transgene DNA was co-injected into the blastomere of the one-cell stage embryo with 100 pg mRNA encoding the Tol2 transposase. F0 fish were raised to adulthood and intercrossed. F1 progeny were screened for GFP fluorescence in the margin at 30-50% epiboly, or in the Kupffer's vesicle in the early somite stages.

Activation and Inhibition of the Nodal Pathway

To activate the Nodal pathway, TARAM-D was expressed, which is a mutated and constitutively activated form of the Nodal receptor (Renucci et al., Development, 122; 3735-3743 (1996). Capped mRNA was synthesized using the Ambion mMessage mMachine™Kit (Ambion, Inc., Austin, Tex.). 100 pg mRNA was injected into 1- to 2-cell-stage embryos. To block the Nodal receptors, SB-505124 was used, which is a specific inhibitor of the ALK 4,5,7 receptors and has been previously demonstrated to phenocopy embryos lacking nodal function (DaCosta Byfield et al., Mol. Pharmacol., 65: 744-752 (2004); Hagos and Dougan, BMC Dev. Biol., 7: 22 (2007).

In Situ Hybridization and Sections

In situ hybridization was performed as previously described (Dougan et al., Development, 130: 1837-1851 (2003). Embryos were processed to reveal expression of green fluorescent protein (gfp) (Cormack et al., Gene, 173: 33-38 (1996), goosecoid (gsc) (Stachel et al., Development, 117: 1261-1274 (1993), floating head (flh) (Talbot et al., Nature, 378: 150-157 (1995), no-tail (ntl) (Schulte-Merker et al., Development, 120: 1009-1015 (1994), myosin17 (my17/cardiac light chain myosin-2) (Yelon, Dev. Dyn., 222; 552-563 (2001), mezzo (Poulain and Lepage, Development, 129: 4901-4914 (2002), sox17 (Alexander and Stainier, Biol., 9: 1147-1157 (1999), sqt (Feldman et al., Nature, 395: 181-185 (1998) and cyc (Rebagliati et al., Proc. Natl. Acad. Sci. U.S.A., 95: 9932-9937 (1998b); Sampath et al., Nature, 395: 185-189 (1998). Following in situ hybridization, selected embryos were dehydrated in a series of 100% methanol, 100% ethanol, acetone and propylene oxide. Embryos were then incubated overnight in a 1:1 mixture of propylene oxide: Epon-Araldite, and for 1 h in a 1:2 mixture of propylene oxide: Epon-Araldite. Following two washes in 100% Epon-Araldite, the resin was allowed to polymerize at 70° C. for at least 18 h. Embryos were then cut into 3-μm sections and mounted in Permount (Sigma-Aldrich, Inc., St. Louis, Mo.).

### Example 1

### Isolation of sqt Regulatory Sequences

The strict spatio-temporal regulation of nodal-related gene expression is essential for germ layer formation (Schier, Cell Dev. Biol., 19: 589-621 (2003). In order to understand the molecular mechanisms controlling nodal-related gene expression in zebrafish, a 120 kb BAC clone containing the entire sqt genomic locus was identified. A BAC recombineering strategy was used to replace the first exon of sqt with the eGFP open reading frame (FIG. 1A) (Lee et al., Genomics 73: 56-65 (2001). After transient expression of the manipulated BAC clone, GFP fluorescence was observed at the margin in blastula stage embryos (5 hpf; FIG. 1C) and in a few dorsally located cells during gastrulation (FIG. 1D, arrow). This indicates that the BAC contains all the genomic sequences necessary to drive reporter gene expression in the marginal blastomeres and the dorsal forerunners.

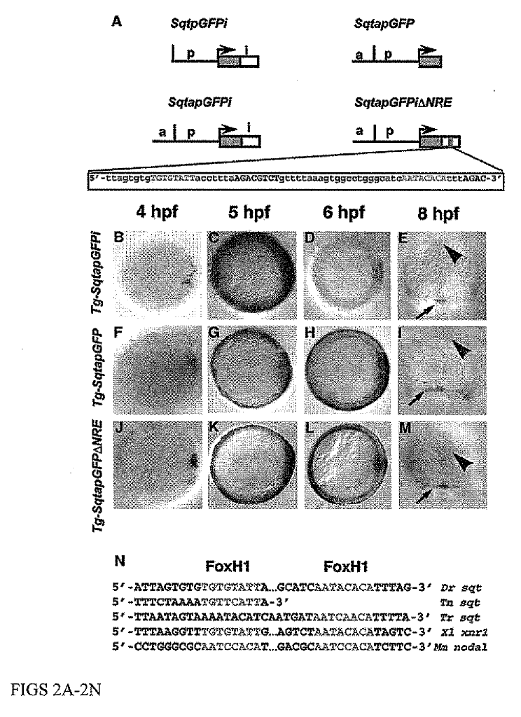

To further narrow down the region necessary for sqt expression, the gap-repair method was used to generate a smaller clone from the BAC, which consisted of 9.9 kb of genomic sequences upstream of gfp and 603 bp of the first introns downstream of gfp (FIG. 1B; p9.9sqtGFPi) (Liu et al., Genome Res., 13: 476-484 (2003). After injection, GFP fluorescence was observed in the margin (FIG. 1E) and in the forerunner cells (data not shown). This region contains enhancers responsible for driving sqt expression in the margin. To define a smaller region capable of driving GFP expression in the sqt pattern, p9.9sqtGFPi was subdivided into six fragments using convenient restriction sites (FIG. 1B). A fragment containing only the proximal 923 bp of upstream genomic sequences and the 603 bp intron did not express GFP, as indicated by the lack of fluorescence in living embryos at the blastula stage or later stages (FIG. 2A, construct SqtapGFPi; data not shown). Next, the upstream fragments were investigated to determine which of them contained the sequences necessary for sqt expression. GFP fluorescence was observed when the distal-most ‘a’ fragment was inserted upstream of the proximal fragment (FIG. 2A, construct apGFPi, data not shown), but not when any of the other fragments were inserted at this position. This distal fragment is not capable of driving GFP expression when fused to a heterologous promoter (see Materials and methods; data not shown). Thus, neither the ‘a’ fragment nor the proximal fragment alone is sufficient to drive expression of a reporter gene in the margin of blastula stage embryos. This indicates that sequences in both fragments are necessary for expression in the margin. These results do not rule out the possibility that other important regulatory sequences are contained within the large BAC clone.

The temporal expression of sqt is controlled by sequences in the first introns The inherent variability and mosaicism of transiently expressed transgenes complicates analysis of their expression (Hsiao et al., Dev. Dyn., 220: 323-336. (2001). To circumvent these problems, the Tol2 transposon was used to generate a stable transgenic line containing both the distal and proximal fragments upstream of gfp and 603 bp of the first intron downstream (FIG. 2A; SqtapGFPi) (Kawakami et al., Dev. Cell 7: 133-144 (2004); Urasaki et al., Genetics, 174: 639-649 (2006). Despite relatively low fluorescence intensity of GFP protein at early stages, gfp mRNA was easily detected by in situ hybridization in all three phases of sqt expression (FIG. 2). Soon after MBT, gfp transcripts are detected in a few dorsal blastomeres (FIG. 2B). Like sqt, gfp is expressed in a marginal ring at 5 hpf and is down-regulated in the margin at 6 hpf (FIGS. 2C, D). Transgene expression persists in the dorsal forerunner cells throughout gastrulation (FIG. 2E). Thus, Tg-SqtapGFPi recapitulates the major features of the endogenous sqt spatiotemporal expression pattern. This indicates that the factors that control sqt expression in each of its phases also act upon the transgene. Low levels of gfp expression in the axial mesoderm were observed in each of the three lines, but do not reflect endogenous sqt expression (FIGS. 2E, I, M, arrowheads). This raises the possibility that the construct lacks an element responsible for repressing sqt expression in the axial mesoderm.

Because important regulatory sequences have been identified in the first introns of nodal-related genes in Xenopus and mice, whether the sqt intron contains essential regulatory elements was investigated Norris et al., Development, 129: 3455-3468 (2002); Osada et al., Development, 127: 2503-2514 (2000). A second line was generated that lacks the intron sequences, but retains both the distal and proximal upstream fragments (FIG. 2A; SqtapGFP). In this line, gfp expression initiates soon after MBT (FIG. 2F), as does endogenous sqt (Erter et al., Dev. Biol., 204: 361-372 (1998); Feldman et al., Nature, 395: 181-185 (1998). At 5 hpf, a ring of gfp expression was observed in the margin (FIG. 2G). Finally, at the onset of gastrulation, gfp was expressed in the dorsal forerunner cells and continues throughout gastrulation (FIG. 2I and data not shown). The distal and proximal sqt genomic fragments upstream of GFP contain enhancer elements responsible for inducing sqt expression in the dorsal blastomeres, the embryo margin and the dorsal forerunner cells.

Consistent with this, three putative Tcf/Lef binding sites were found in the 923 bp upstream of the sqt transcription start site, which are included in the transgene and could mediate activation of gene expression by β-catenin (data not shown) (Dorsky et al., Dev. Biol., 241: 229-237 (2002). By contrast, only one putative Tcf/Lef site was found in the region between −900 bp and −1800 bp upstream of the sqt transcription start site, suggesting that the clustering of sites close to the sqt transcription start site may be significant. The factors controlling sqt expression in the marginal ring have not been identified.

### Example 2

### Distinct Mechanisms Control Sqt Expression in Embryonic and Extra-Embryonic Tissues

Reporter gene expression in Tg-SqtapGFP differed from g expression in Tg-SqtapGFPi and endogenous sqt expression in two ways. Firstly, the ring of gfp expression in Tg-SqtapGFP persists in marginal cells throughout gastrulation (FIGS. 2H, I; compare with FIGS. 2D, E). This indicates that sequences in the sqt first intron are required to repress gene expression at the margin during gastrulation. Secondly, gfp expression appeared narrower in Tg-SqtapGFP than in Tg-SqtapGFPi, and seemed confined to a more superficial layer of cells (FIGS. 2C, G). To test this, gfp expression was analyzed in sections of 5 hpf embryos from both lines (FIGS. 3A, B). In Tg-SqtapGFPi, gfp is expressed in the YSL (FIG. 3A, under red line), blastomeres (FIG. 3A, above the red line) and EVL at roughly equivalent levels (FIG. 3A, arrow). This accurately reflects the expression pattern of sqt. In the absence of the intron, by contrast, gfp is strongly expressed in the YSL (FIG. 3B, under red line) and EVL (FIG. 3B, arrow), but is not detected in the blastomeres. The expression in the EVL appears as a narrow ring in whole mounts (FIG. 20). These results indicate that genetically distinct pathways control sqt expression in the embryonic and extraembryonic tissues. Rare blastomeres do express gfp, and these are invariably adjacent to the YSL or EVL (FIG. 3B, above red line). This staining could indicate that rare blastomeres in 5 hpf embryos share the regulatory program of the extra-embryonic tissues. The intron contains a tissue specific enhancer responsible for driving expression in the blastomeres. This is consistent with results demonstrating that nodal-related genes in mice and frog contain conserved enhancers that drive tissue specific expression in the LLPM and boost nodal levels in the frog margin and mouse epiblast (Adachi et al., Genes Dev., 13: 1589-1600 (1999); Brennan et al., Nature, 411: 965-969 (2001); Hyde and Old, Development, 127: 1221-1229 (2000); Osada et al., Development, 127: 2503-2514 (2000); Saijoh et al., Dev. Biol., 256: 161-173 (2003).

To understand the pathway that controls sqt expression in the blastomeres, whether the intron mediates the response to Nodal signals was investigated. Embryos were injected from both lines with mRNA encoding a mutated and constitutively activated version of the Nodal receptor, called TARAM-D (Renucci et al., Development, 122: 3735-3743 (1996). This receptor acts in a cell autonomous manner to dorsalize embryos and induce expression of Nodal target genes (Aoki et al., Dev. Biol., 241: 273-288 (2002); Renucci et al., Development, 122: 3735-3743 (1996). In response to excess Nodal signaling, the blastomeres adopt dorsal mesendodermal fates and the embryos fail to undergo epiboly (Hagos and Dougan, BMC Dev. Biol., 7: 22 (2007); Shimizu et al., Mech. Dev., 91: 293-303 (2000). This accounts for the lack of doming and dramatically altered morphology of the YSL in TARAM-D injected embryos (FIGS. 3E, F). In addition, EVL cells lose their normal squamous shape and become rounded (compare FIG. 3B, arrow and FIGS. 3E, F, arrows). This indicates that EVL cells are capable of responding to activation of the Nodal pathway. If the intron is required to respond to Nodal signaling, then TARAM-D should induce ectopic gfp expression in Tg-SqtapGFPi, but not in the line lacking the intron. In contrast to this expectation, both lines exhibited a response to TARAM-D as indicated by a dramatic expansion of gfp expression apparent in whole mounts (FIGS. 3C, D). The responses of the two lines are not equivalent, however, as revealed by sectioning embryos expressing TARAM-D. In Tg-SqtapGFPi embryos expressing TARAM-D, gfp is expressed in blastomeres throughout the entire embryo including all blastomeres and EVL cells and the YSL (FIG. 3E). gfp is expressed at roughly equivalent levels in the YSL and blastomeres. The reduced level of expression in the internal cells is likely due to the poor access of these cells to in situ reagents. Because there was no change in the pattern of gfp expression in the YSL, it could not be determined if the YSL responds to TARAM-D. Tg-SqtapGFPi contains all the sequences necessary for blastomeres and EVL cells to respond to Nodal signals. By contrast, in the line lacking the intron, gfp expression expands throughout the EVL in response to TARAM-D (FIG. 3F, arrows), but is not detected in the blastomeres (FIG. 3F). Thus an upstream enhancer mediates the response to Nodal signals specifically in the EVL. Thus, the Nodal signaling pathway acts by distinct mechanisms to induce transgene expression in the blastomeres and the EVL. These results raised the possibility that gfp expression in the marginal blastomeres and EVL of Tg-SqtapGFPi embryos depends on Nodal signals. To test this, the chemical inhibitor of the Nodal receptors, SB-505124, was utilized to block Nodal signaling in Tg-SqtapGFPi embryos (DaCosta Byfield et al., Mol Pharmacol., 65: 744-752 (2004); Hagos and Dougan, BMC Dev. Biol., 7: 22 (2007). 50 μM of this compound is sufficient to phenocopy embryos lacking nodal-related gene function when added to embryos at 3 hpf or earlier, whereas later treatments, or lower doses, generate milder phenotypes (Hagos and Dougan, BMC Dev. Biol., 7: 22 (2007). When Tg-SqtapGFPi embryos were treated with 50 μM SB-505124, overall levels of gfp expression are reduced, but they are reduced to a greater extent in the blastomeres and EVL than in the YSL (FIG. 3G). In some embryos, expression was seen in cells in the area of the EVL, but this probably represents expression in the dorsal forerunner cells (data not shown). In the YSL, gfp expression is consistently reduced, but not completely eliminated (FIG. 3G). This demonstrates that gfp expression in the blastomeres and EVL depends on activation of the Nodal-signaling pathway.

### Example 3

### The sqt First Intron Contains an Essential Nodal Response Element

Two sequences were found in the sqt first intron that fit the FoxH1 consensus-binding site, TGT(T/G)(T/G)ATT (SEQ ID NO: 17), as defined by in vitro binding assays (FIG. 2A, SqtapGFPiΔNRE, red letters) (Zhou et al., Mol Cell, 2: 121-127 (1998). This is similar to the “paired FAST” site defined in Xenopus (FIG. 2N) (Osada et al., Development, 127: 2503-2514 (2000). These sites are located in close proximity to at least three core Smad binding sequences, AGAC (FIG. 2A, SqtapGFPiΔNRE, black bold letters) (Zawel et al., Mol. Cell, 1: 611-617 (1998). The introns of sqt orthologues in the pufferfish Tetraodon nigroviridis and Takifugu rubripes each contain a single putative FoxH1 binding site (FIG. 2N) (Aparicio et al., Science, 297: 1301-1310 (2002); Jaillon et al., Nature, 431: 946-957 (2004). Since an estimated 200 Myr of evolution separates pufferfish from zebrafish, this shows a strong conservation of the intronic enhancer throughout the teleost lineage (Taylor et al., Trans. R. Soc. Lond., B Biol. Sci., 356: 1661-679 (2001).

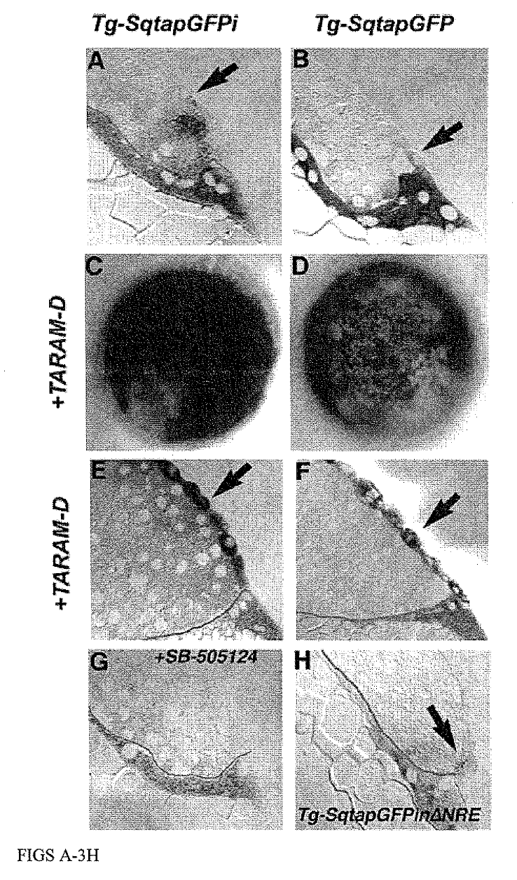

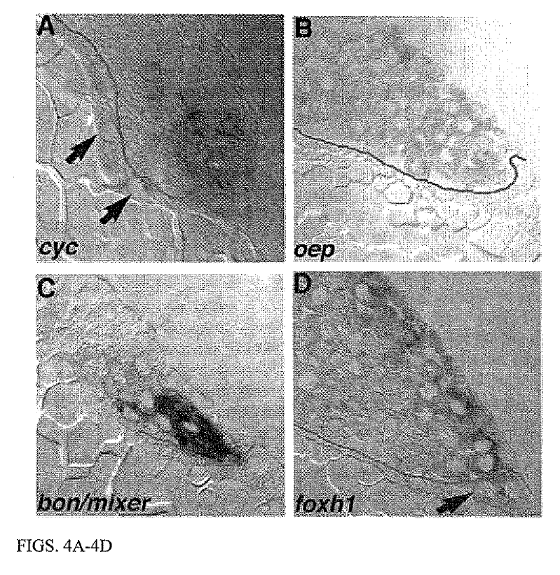

To test whether this putative element is required for gfp expression in the blastomeres, a transgenic line was generated lacking both FoxH1 sites and associated Smad sites in the introns (FIG. 2A; Tg-SqtapGFPiΔNRE). In this line, gfp expression initiates in the dorsal blastomeres soon after MBT (FIG. 2J). In the late blastula stage (5 hpf), gfp is expressed in a narrow ring at the margin and strongly resembles reporter gene expression in YSL has already been documented (Erter et al., Dev. Biol., 204: 361-372 (1998); Feldman et al., Nature, 395: 181-185 (1998). To determine if cyc is expressed in the YSL, late blastula stage embryos were sectioned (5 hpf) that were stained to reveal the localization of cyc transcripts. cyc is expressed in marginal blastomeres, up to 6 tiers from the boundary of the YSL (FIG. 4A). Transcripts are concentrated in the perinuclear space as expected for a secreted protein, giving the stain a punctate appearance. cyc mRNA around the nuclei of the YSL (FIG. 4A, arrows). This demonstrates that the YSL is a source of both Sqt and Cyc signals that could act upon the overlying blastomeres.

The results also suggest the possibility that Nodal signals in the blastomeres signal to the EVL and, perhaps, to the YSL. If so, then components of the Nodal signal transduction pathway should be expressed in the YSL and EVL. Therefore, expression of the Oep co-receptor was examined, and the FoxH1 and Bon/Mixer transcription factors in sections of mid-blastula stage (4 hpf to 4.3 hpf) embryos. Despite the fact that maternal and zygotic oep transcripts are ubiquitously distributed in early embryos, oep mRNA is excluded from the YSL by 4 hpf (FIG. 4B) (Zhang et al., Cell, 92: 241-251 (1998). Interestingly, oep transcripts are found in all EVL cells (FIG. 4B). By contrast, bon/mixer transcripts are excluded from the EVL and YSL at 4.3 hpf (FIG. 4C). bon/mixer mRNA is found exclusively in the marginal blastomeres, within 3-4 tiers of the YSL (FIG. 4C). At the same stage, foxh1 is expressed in all blastomeres as well as the YSL (FIG. 4D, arrow). Levels of foxh1 transcripts are elevated in the EVL and the layer of blastomeres immediately beneath the EVL (FIG. 4D). foxh1 is also expressed at lower levels in the interior blastomeres (FIG. 4D). It is not known if this difference in expression levels has functional significance, or if it reflects the greater accessibility of the superficial cells to the reagents for in situ hybridization. Nonetheless, the expression of Oep and FoxH1 in the EVL indicates that these cells are competent to respond to Nodal signals. Although the lack of oep transcripts in the YSL suggests that this tissue is not competent to respond to Nodal signals, it remains possible that Oep protein is produced from transcripts localized in this tissue at earlier stages.

### Example 4

### Sqt and Cyc Signals in the YSL are Required to Pattern the Embryo

To test if sqt and cyc are required in the YSL, their function in this tissue was specifically knocked down by targeted injection of antisense morpholinos (MOs) directed against sqt and cyc transcripts. Translation-blocking MOs against sqt and cyc have been described previously (Feldman and Stemple, Genesis, 30:175-177 (2001); Karlen and Rebagliati, Genesis, 30:126-128 (2001). Experiments with fluorescently labeled MOs showed that they do not diffuse across cell membranes and act exclusively in a cell autonomous manner (Amack and Yost, Curr. Biol. 14: 685-690 (2004). To confirm this, the distribution of a lissamine-tagged control MO was examined in live embryos 2 h and 24 h after injection at 3 hpf (FIGS. 5A, B). After 2 h, the MO is distributed in a ring underneath the blastomeres, indicating that the MO is restricted to the YSL at this stage (FIG. 5A). An identical distribution was observed when fluorescently tagged Dextran was injected into the YSL (Feldman et al., Nature, 395: 181-185 (1998). At 24 hpf, the fluorescence is still restricted to the yolk, demonstrating that the MO does not diffuse across the membrane into the blastoderm (FIG. 5B). Next, 8 ng each of fluorescently tagged MOs against sqt and cyc was injected, or mismatched controls, directly into the YSL soon after it formed at 3 hpf Embryos injected with both control MOs have normal embryonic shields at 6 hpf (100%, N=24) (FIG. 5C, arrow), and develop a normal body axis at 24 hpf, with a notochord, somites and heart (100%, N=18) (FIG. 5D). When cyc function alone is depleted from the YSL, all embryos have normal shields (100%, N=36) (FIG. 5E, arrow). At 24 hpf, the cyc-depleted embryos, are indistinguishable from embryos injected with control MOs (100%, N=22) (FIG. 5F). When sqt function is depleted from the YSL, by contrast, the majority of embryos lack the embryonic shield (54%, N=11) (FIG. 5G). Previous results demonstrated that the organizer does not form in embryos lacking zygotic sqt function (Dougan et al., Development, 130: 1837-1851 (2003); Feldman et al., Nature, 395: 181-185 (1998). The results extend these previous studies by demonstrating that sqt function is required in the YSL to induce the morphological shield.

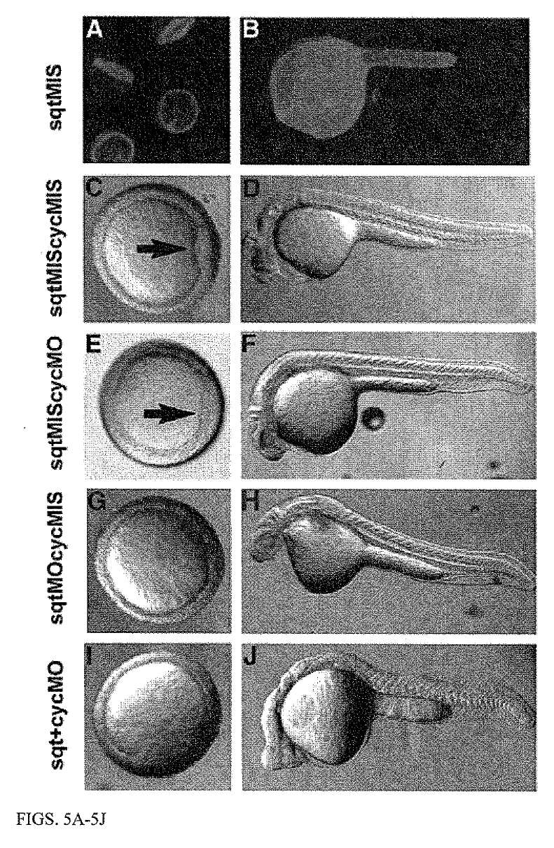

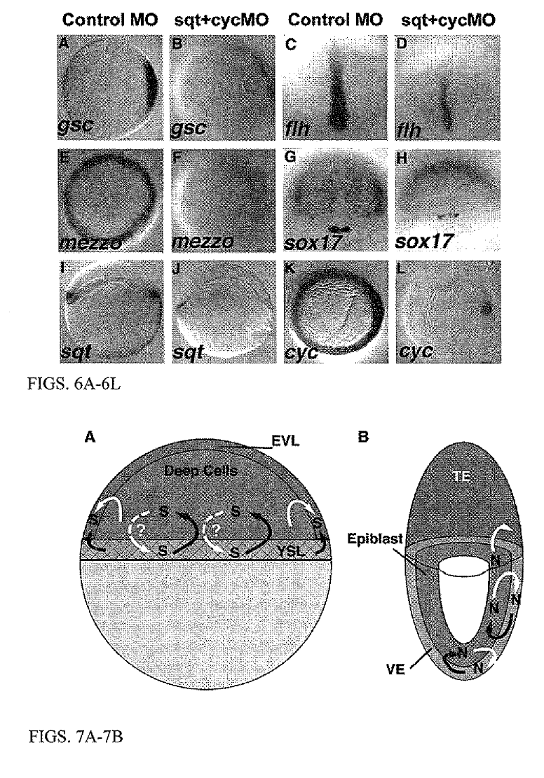

At 24 hpf, a distinct minority of embryos lacking sqt function in the YSL displayed mild cyclopia, indicating that a substantial fraction of the defective embryos at 6 hpf have recovered (16%, N=54) (FIG. 5H). This is consistent with the observation that sqt mutants recover during gastrulation in a cyc-dependent manner (Dougan et al., Development, 130: 1837-1851 (2003); Feldman et al., Nature, 395: 181-185 (1998); Hagos and Dougan, BMC Dev. Biol., 7: 22 (2007). This raised the possibility that Cyc signals in the YSL could compensate for the loss of Sqt in the YSL. To test this, both sqt and cyc function was simultaneously depleted from the YSL. All of these embryos have reduced or missing shields at 6 hpf(100%, N=23) (FIG. 5I) and the majority lacked gsc expression at 5 hpf indicating dorsal mesoderm is not specified at this stage (FIG. 6B, 71%, N=28). These embryos also lack endoderm, as indicated by the absence mezzo and sox17 expression (FIGS. 6E-H) (Alexander and Stainier, Curr. Biol., 9: 1147-1157 (1999); Poulain and Lepage, Development, 129: 4901-4914 (2002). By 24 hpf the majority of these embryos display severe cyclopia and have narrow notochords (70%, N=20) (FIG. 5J). Consistent with this, flh expression is reduced during gastrulation (FIG. 6D) (Talbot et al., Nature, 378: 150-157 (1995). These results demonstrate that sqt and cyc have partially overlapping functions in the YSL and are required to induce the organizer and head mesoderm and endoderm.

### Example 5

### Sqt and Cyc Signals in the YSL are Required to Induce Nodal-Related Gene Expression

The analysis of sqtGFP transgenes suggested that Nodal signals in the YSL induce or maintain sqt expression in the overlying blastomeres via the conserved NRE in the first intron. Whether Nodal signals in the YSL are required for sqt and cyc expression in the embryo was investigated. When the two genes are simultaneously depleted from the YSL, expression of sqt (93%, N=55) (FIGS. 6I, J) and cyc (63%, N=49) (FIGS. 6K, L) are reduced in the majority of the embryos. Low levels of sqt expression are detected in these embryos, but these transcripts are located predominantly in the YSL (FIG. 6J), where the MO prevents translation of sqt mRNA. sqt transcripts were not detected in the marginal blastomeres or in the EVL indicating that endogenous sqt, like the transgene, is controlled by a different regulatory program in the YSL than in the blastomeres. cyc is expressed in a few marginal blastomeres when Nodal signals are depleted from the YSL (FIG. 6L). Although the dorso-ventral axis is not apparent at this stage, it was previously demonstrated that dorsal expression of cyc is independent of earlier Nodal signals (Hagos et al., Dev. Biol., (2007). Therefore in these experiments, it is likely that cyc is expressed in dorsal blastomeres. This domain of cyc expression accounts for the formation of trunk mesoderm in embryos lacking extraembryonic Nodal signals. These results demonstrate that Nodal signals from the YSL are required for nodal-related gene expression in the overlying blastomeres.

Discussion

In this work, the first analysis of the regulatory elements that control nodal-related gene expression in zebrafish is presented. Zygotic sqt is expressed in three independent phases. A 1.9-kb region of DNA was identified that is sufficient to drive reporter gene expression in the endogenous sqt spatio-temporal expression pattern. This artificial promoter is comprised of three fragments from non-contiguous regions in the endogenous gene, including a 431-hp distal element located 9.4 kb upstream of the sqt transcription start site, the 923 bp directly upstream of the sqt transcription start site and 603 bp of the first intron. Since these sequences contain the cis-acting elements necessary to drive normal expression of sqt, the roles of these sequences were analyzed with the goal of better understanding the molecular mechanisms that control sqt expression in each of its phases. Although the transgenes accurately reflect many aspects of the endogenous sqt expression pattern, gfp expression behaved differently than endogenous sqt in some experiments. In the absence of Nodal signaling, for example, gfp expression persists in the YSL at a stage when endogenous sqt is not detected (FIG. 3G) (Meno et al., Mol. Cell, 4: 287-298 (1999). It is likely that this is due greater sensitivity of the gfp probe, as compared to that of the sqt probe. Supporting this idea, gfp expression was detected in dorsal blastomeres as early as 2.5 hpf, a half an hour before the earliest reported expression of sqt (3.0 hpf) (data not shown) (Feldman et al., Nature, 395: 181-185 (1998). It is possible that the transgenes lack some elements necessary to fully recapitulate the endogenous pattern of sqt expression.

sqt is Expressed in Three Independently Controlled Temporal Phases.

The intron is not required for transgene expression in the dorsal blastomeres before 4 hpf (FIG. 2F). This indicates that the sequences mediating the first phase of sqt expression are located in one of the two fragments upstream of the gfp coding region. Previous genetic analysis demonstrated that this expression depends on the dorsal determinant, β-catenin, but it is not known if sqt is a direct target (Bellipanni et al., Development, 133: 1299-1309 (2006); Dougan et al., Development, 130: 1837-1851 (2003). At least three potential binding sites for the β-catenin binding partner were found, Lef1/Tcf in the 923 bp upstream of the transcription start site, and an additional site in the distal fragment (data not shown) (Dorsky et al., Dev. Biol., 241: 229-237 (2002). The data is consistent with the idea that sqt is a direct target of β-catenin. The element driving sqt expression in the dorsal forerunners during gastrulation must also be present in the sequences upstream of the gfp coding region, since all of the transgenes expressed gfp in the dorsal forerunners (FIGS. 2E, IM). This enhancer remains to be identified.

Little is known about the factors that control expression of sqt at the margin during the blastula stages. This phase of sqt expression is particularly important, since studies conditionally inactivating the Nodal receptors show that Nodal signals are most active in patterning the germ layers during the mid-to-late blastula stages (3.5-5 hpf) (Hagos and Dougan, BMC Dev. Biol., 7: 22 (2007). Expression at this stage is independent of the earlier expression of sqt in the dorsal blastomeres, since depletion of β-catenin eliminates the early dorsal expression of sqt but does not affect sqt expression in the marginal ring (Bellipanni et al., Development, 133: 1299-1309 (2006); Kelly et al., Development, 127: 3899-3911 (2000). The data indicates that proper expression of sqt at this stage involves the complex interaction of three different cell types.

sqt is Expressed Independently in Three Tissues in the Margin.

In the late blastula stage, sqt is expressed in three distinct marginal tissues, including the marginal blastomeres and the extra-embryonic YSL and EVL (Erter et al., Dev. Riot, 204: 361-372 (1998); Feldman et al., Nature, 395: 181-185 (1998); Rebagliati et al., Dev. Biol., 199: 261-272 (1998a). The data demonstrates that expression in each tissue is controlled by separable elements. Firstly, expression in the blastomeres is mediated by a conserved Nodal response element (NRE) within the first sqt intron (FIG. 3H). gfp expression in the blastomeres depends upon Nodal signaling (FIG. 3G), and the response to activation of the Nodal pathway in the blastomeres is mediated entirely by sequences in the first intron (FIG. 3F). Similarly, expression of the transgenes and endogenous sqt in the EVL depend upon Nodal signaling (FIGS. 3G and 6J). Surprisingly, introns sequences are dispensable for expression in this tissue (FIG. 3B). This indicates that an element in one of the two fragments upstream of gfp is capable of mediating the response to Nodal signals in the EVL. Consistent with this idea, there are three FoxH1 consensus sites in the 923 bp directly upstream of gfp in the transgenes (X. Fan and S. Dougan, unpublished observations). These sites could mediate the EVL response, but interestingly, they are not capable of mediating the response to TARAM-D in blastomeres (FIG. 3F). This indicates that there are significant functional differences between the FoxH1 sites, but it remains to be determined why the intron NRE mediates expression in the blastomeres and the upstream FoxH1 sites do not. The other known effector of the Nodal signaling pathway, Bon/Mixer, cannot mediate the response to Nodal signals in the EVL since it is not expressed in this tissue (FIG. 4C).

Finally, expression in the YSL is controlled by sequences upstream of gfp, but it is not clear if these sequences mediate a response to the Nodal-signaling pathway in the YSL like they do in the EVL (FIGS. 3B, G). When Nodal signaling is reduced, expression of gfp in the blastomeres is reduced to a much greater extent than in the YSL (FIG. 3G). Similarly when embryos are depleted of extra-embryonic Nodal signals, endogenous sqt persists in the YSL after expression in the blastomeres and EVL is no longer detected (FIG. 6J). Both gfp and sqt are eventually lost from the YSL when Nodal signals are blocked, although gfp expression persists longer than sqt expression. The loss of gfp and sqt from the YSL could be due to decreased expression of an inducer of nodal-related gene expression, or to increased expression of an inhibitor. Unlike most other nodal-related genes, sqt is not expressed in the left lateral plate mesoderm (LLPM) after gastrulation (Erter et al., Dev. Biol., 204: 361-372 (1998); Feldman et al., Nature, 395: 181-185 (1998); Rebagliati et al., Dev. Biol., 199: 261-272 (1998a). Despite this, the sqt NRE is similar to the intronic asymmetric enhancer (ASE) that drives expression in the LLPM of ascidians, frogs and mammals (FIG. 2N) (Hyde and Old, Development, 127: 1221-1229 (2000); Osada et al., Development, 127: 2503-2514 (2000). This suggests that the sqt NRE lacks critical sequences necessary for asymmetric expression. The ASE is comprised of two FoxH1 sites flaming a conserved core “TTG(G/C)CCA” (SEQ ID NO:17) motif (Osada et al., Development, 127: 2503-2514 (2000). The sqt NRE contains two FoxH1 consensus sites, but lacks the conserved core (FIG. 2A). This raises the possibility that the TTG(G/C) CCA sequence binds the transcription factor complex that drives expression in the LLPM. It remains possible that the sqt NRE lacks other sequences that drive asymmetric expression.

Nodal Signals Mediate Interactions Between Embryonic and Extra-Embryonic Tissues.

Nodal signals are required during the mid-to-late blastula stages, a period of rapid cell division and cell intermixing (Hagos and Dougan, Dev. Biol., 7: 22 (2007). This raises the question of how a stable zone of Nodal signaling is maintained within such a dynamic cell population. YSL is a source of Nodal signals that are independent of the population of overlying blastomeres. First, expression of the transgene in the YSL is controlled by a different element than the one that controls expression in the blastomeres. Second, expression of a sqt transgene in the blastomeres is reduced when Nodal signaling is blocked with SB-505124, but expression in the YSL persists (FIG. 3G). Third, cyc is expressed in the YSL (FIG. 4A), along with sqt (Erter et al., Dev. Biol., 204: 361-372 (1998); Feldman et al., Nature, 395: 181-185 (1998). Finally, sqt and cyc expression in the blastomeres depends, in part, upon Nodal signaling in the YSL (FIGS. 61-L). Depletion of Sqt and Cyc signals in the YSL causes an overall reduction in Nodal signaling levels in the entire embryo. This in turn, results in a loss of endoderm and dorsal mesoderm (FIGS. 5 and 6). Thus the YSL is a stable source of Nodal signals that act to impose a reproducible pattern upon a dynamic population of overlying blastomeres. The YSL must express other essential signals, however, since the defects resulting from degrading all mRNA in the YSL are much more severe than those from specific depletion of Sqt and Cyc (Chen and Kimelman, Development, 127: 4681-4689 (2000). Nodal signals in the EVL may also act to stabilize expression in the blastomeres, but with the currently available technology, it is not possible to deplete sqt and cyc function specifically in the EVL.

The results suggest a model in which sqt expression in the margin is induced in the YSL by maternal transcription factors acting on an element upstream of the sqt transcription start site. Genetic data presented here and in previous studies indicates that cyc expression in the YSL must be induced independently of sqt function, otherwise Cyc signals could not compensate for the depletion of Sqt in the YSL (FIG. 5) (Dougan et al., Development, 130: 1837-1851 (2003); Feldman et al., Nature, 395: 181-185 (1998). Sqt and Cyc signals from the YSL subsequently induce sqt expression in the blastomeres, acting through the NRE in the first intron (FIG. 7A). Sqt and Cyc signals also act through an upstream response element to induce sqt expression in the EVL (FIG. 7A). It is not known if Nodal signals from the YSL can directly induce sqt expression in the EVL, or if Nodal signals from the blastomeres are required for sqt expression in the EVL. Both tissues may act as sources, however, given the proximity of the EVL cells to the YSL and marginal blastomeres (FIG. 7A). Further experiments are necessary to determine if similar mechanisms control expression of cyc in these tissues.

The interactions between the YSL, EVL and marginal blastomeres serve to create a stable source of Nodal signaling at the margin despite the rapid cell divisions and intermixing that occurs during the blastula stages (Kimmel and Law, Dev. Biol., 124: 269-280 (1985); Kimmel and Warga, Dev. Biol., 124, 269-280 (1987). If a cell moves away from the margin beyond the range of Nodal signals, then it will stop expressing sqt or cyc. By contrast, sqt and cyc expression will be induced if a cell moves to a position within the range of Nodal signals at the margin. Since nodal-related gene expression is not simultaneously induced in the YSL and overlying blastomeres, there is a temporal gradient of sqt expression along the animal-vegetal axis. Cells that remain close to the YSL express sqt for a longer period than cells located farther from the margin. The length of time cells express sqt and cyc could have profound consequences on their eventual cell fate choice.

The Conserved Roles of Teleost and Mammalian Extra-Embryonic Tissues.

The interaction between embryonic and extra-embryonic tissues in zebrafish is remarkably similar to those previously described in other vertebrates. Previous groups have recognized that the mammalian visceral endoderm, teleost YSL and chick hypoblast each express orthologues of the Hex transcription factor (Ho et al., Curr. Biol., 9: 1131-1134 (1999); Martinez Barbera et al., Development, 127: 2433-2445 (2000); Yatskievych et al., Mech. Dev., 80; 107-109 (1999). This has led to the suggestion that these tissues share a common evolutionary origin (Ho et al., Curr. Biol., 9: 1131-1134 (1999). Nodal signals in the zebrafish YSL are required for head mesoderm and endoderm (FIGS. 5 and 6). Similarly Chimeric mutant mice lacking nodal function in the visceral endoderm have defects in the prechordal plate, which disrupts anterior neural development (Varlet et al., Development, 124; 1033-1044 (1997). Secondly, in both species a conserved intronic NRE mediates the response to Nodal signals in the embryo (FIGS. 2 and 3) (Brennan et al., Nature, 411: 965-969 (2001). In the mouse, elimination of the NRE reduces, but does not eliminate, expression of the transgene in the epiblast (Brennan et al., Nature, 411: 965-969 (2001). Thus, Nodal signals in the YSL act by the same mechanism as those in the visceral endoderm to induce the prechordal plate. This strengthens the argument that the tissues share a common evolutionary origin. The fact that gfp expression in the YSL persists longer than it does in the blastomeres when Nodal signals are blocked suggests that expression of sqt in the YSL does not directly depend upon Nodal signaling (FIG. 3G). This idea is strengthened by the observation that sqt expression persists in the YSL, but not in blastomeres, when extra-embryonic Nodal signals are depleted (FIG. 6J).

One major difference between the species is that in the mouse, Nodal signals from the epiblast induce nodal expression in the visceral endoderm (FIG. 7B) (Brennan et al., Nature, 411: 965-969 (2001). By contrast, it is not clear if the teleost YSL responds to Nodal signals from the blastomeres. The Nodal effector, FoxH1 is expressed in the YSL, but oep mRNA is excluded from the YSL from an early stage (FIGS. 4B, 1)). Since maternal oep is ubiquitously expressed, this suggests that maternal oep transcripts are rapidly cleared out of the YSL (Zhang et al., Cell, 92: 241-251 (1998). Injecting oep MOs into the YSL produced no phenotype (E. Hagos, B. Xu, R. Burdine, S. Dougan, unpublished observations). It remains possible, however, that Oep protein is still present, or that Nodal signals in the blastorneres induce other signals that act in an Oep-independent manner to maintain nodal-related gene expression in the YSL.

The results also reveal striking parallels between the teleost EVL and the mammalian extra-embryonic ectoderm. In mice, the extra-embryonic ectoderm is derived from the trophectoderm, the outer layer of cells that form during compaction (Rossant, Biol., 15: 573-581 (2004). Similarly, the zebrafish EVL is an extraembryonic tissue that forms during the cleavage stages and encases the cells that produce the embryo (Bouvet, Cell Tissue Res., 170: 367-382 (1976); Kimmel et al., Development, 108: 581-594 (1990). In the mouse, Nodal signals in the epiblast pattern the extra-embryonic ectoderm by a Smad2-independent mechanism, although nodal itself does not appear to be expressed in this tissue (FIG. 7B) (Brennan et al., Nature, 411: 965-969 (2001). Several lines of evidence indicate that the zebrafish EVL is also patterned by Nodal signals. First, EVL cells are competent to respond to Nodal signals since they express the Nodal coreceptor, Oep, and the FoxH1 transcription factor (FIGS. 4B, D). Second, both reporter gene expression and endogenous sqt expression are reduced in the EVL when Nodal signals are blocked by SB-505124 treatment or by loss of extra-embryonic Nodal signals (FIGS. 3G and 6J). This indicates that the EVL cells require Nodal signals, like the mouse extra-embryonic ectoderm. Finally, the expansion of reporter gene expression in the EVL does not depend upon intron sequences (FIG. 3F). This demonstrates that the response to Nodal signals is mediated by a different mechanism in the EVL than in the blastomeres. It has not yet been determined, however, if expression in the EVL is independent of Smad2 function. Nonetheless, the data indicate that in the zebrafish, as in the mouse, the Nodal pathway acts by different mechanisms to pattern different tissues. These parallels raise the possibility that the EVL and extra-embryonic ectoderm share a common evolutionary origin. Further gene expression analysis and functional tests are required to test if this is the case.

The Evolutionary Role of Extra-Embryonic Tissues.

The critical role ascribed to extra-embryonic tissues in patterning the embryo is unique to vertebrates. The tissues that respond to extra-embryonic Nodal signals are characterized by rapid proliferation, extensive cell movements and intermixing. In the zebrafish, the intermixing of the blastomeres during the blastula period has been well documented (Kimmel and Law, Dev. Biol., 108: 94-101 (1985); Kimmel and Warga, Dev. Biol., 124: 269-280 (1987). Cell movements are more extreme in the killifish blastula. In this species, the deep cells that form the embryo migrate in apparently random directions for hundreds of microns during the blastula period, when they respond to patterning signals from the YSL (Oppenheimer, Proc. Natl. Acad. Sci. U.S.A., 20: 536-538 (1934); Trinkaus, Dev. Biol., 30: 69-103 (1973). Similarly, the mouse epiblast is also a dynamic tissue (Tam et al., Microsc. Res. Tech., 26: 301-328 (1993); Varlet et al., Development, 124: 1033-1044 (1997). In contrast to vertebrates, invertebrate chordates, such as amphioxus, completely lack extra-embryonic cells (Tung et al., Sci. Sin., 9: 119-141 (1960). The ascidian Ciona intestinalis does contain extra-embryonic cells, called test cells, but these have a protective function and are not thought to pattern early embryos (Sato and Morisawa, Dev. Genes Evol., 209: 592-600 (1999). Although both ascidians and amphioxus contain migratory cell populations, cells in these embryos do not undergo the extensive intermixing that characterizes many vertebrate tissues (Holland et al., Development, 122: 2911-2920 (1996); Jeffery et al., Nature, 431: 696-699 (2004). The evolution of extra-embryonic sources of patterning signals freed cells to move and intermix to a greater extent than previously possible. This, in turn, may have increased the regulative properties of the embryo, permitting it to restore tissues following insult or injury. Supporting this idea, Xenopus laevis embryos do not have extra-embryonic tissues and do not undergo extensive intermixing during the blastula stages (Dale and Slack, Development, 99: 527-551 (1987). Thus, to a first approximation, the ability of cells to move with respect to the source of Nodal signals correlates with the presence of extraembryonic tissues.

We claim:

1. A transgenic zebrafish expressing a reporter gene under control of sqt regulatory sequences.

2. The transgenic zebrafish of claim 1 wherein the reporter gene encodes a fluorescent protein.

3. The transgenic zebrafish of claim 1 wherein the reporter gene encodes green fluorescent protein.

4. The transgenic zebrafish of claim 1 wherein the sqt regulatory sequences comprise a 431-hp distal element located 9.4 kb upstream of the sqt start site.

5. The transgenic zebrafish of claim 1 wherein the sqt regulatory sequences comprise a 923 bp element located directly upstream of the sqt transcription start site.

6. The transgenic zebrafish of claim 1 wherein the sqt regulatory sequences comprise 603 bp of the first intron.

7. The transgenic zebrafish of claim 1 wherein the sqt regulatory sequences comprise a 431-bp distal element located 9.4 kb upstream of the sqt start site, 923 bp element located directly upstream of the sqt transcription start site, and 603 bp of the first introns.

8. A population of zebrafish embryonic cells comprising a reporter gene under control of sqt regulatory sequences.

9. The population of zebrafish embryonic cells of claim 8 wherein the cells are endodermal cells.

10. The population of zebrafish embryonic cells of claim 8 wherein the cells are mesodermal cells.

11. A method for selecting endodermal or mesodermal embryonic cells comprising dissociating zebrafish embryos comprising a reporter gene under control of sqt regulatory sequences; exposing the cells to an exciting amount of radiation; and collecting the cells that fluoresce.

12. The method of claim 11 wherein the cells are sorted with a fluorescence-activated cell sorter.

13. The method of claim 11 wherein the reporter gene encodes green fluorescent protein.

14. A kit comprising a container comprising a population of zebrafish embryonic cells, wherein the zebrafish embryonic cells comprise a reporter gene under control of sqt regulatory sequences.

15. The kit of claim 14 further comprising cell culture reagents.

16. The kit of claim 14 comprising instructions for using the population of zebrafish embryonic cells.


SEQUENCE LISTING (23 sequences)

SEQ ID NO: 1 - DNA, 25 bp, Artificial Sequence
  Synthetic Morpholino sequence (sqtMO)
atgtcaaatcaaggtaataatccac

SEQ ID NO: 2 - DNA, 26 bp, Artificial Sequence
  Synthetic morpholino sequence (sqtMIS)
atctgaaaatgaagctaataatgcac

SEQ ID NO: 3 - DNA, 24 bp, Artificial Sequence
  Synthetic morpholino sequence (cycMO)
gcgactccgagcgtgtgcatgatg

SEQ ID NO: 4 - DNA, 24 bp, Artificial Sequence
  Synthetic morpholino sequence (cycMO)
gccactgcgagagtgtggatcatg

SEQ ID NO: 5 - DNA, 69 bp, Artificial Sequence
  Synthetic forward primer
cagagactttatttcaataactgcgtgtggattattaccttgatttgacatggtgagcaagggcgagga

SEQ ID NO: 6 - DNA, 70 bp, Artificial Sequence
  Synthetic reverse primer
acttttagcgacgaggctcaagacggagtcagactcgtaaagagttggattctattccagaagtagtgag

SEQ ID NO: 7 - DNA, 30 bp, Artificial Sequence
  Synthetic forward primer
tcatggatccgaagatcaattcaatcccat

SEQ ID NO: 8 - DNA, 29 bp, Artificial Sequence
  Synthetic reverse primer
ttactcgagctaaatcaacgcttagactt

SEQ ID NO: 9 - DNA, 28 bp, Artificial Sequence
  Synthetic forward primer
ttgacctcgctagacgctgtagccttga

SEQ ID NO: 10 - DNA, 31 bp, Artificial Sequence
  Synthetic reverse primer
tccattctagaagtgtttagggcagacaggt

SEQ ID NO: 11 - DNA, 20 bp, Artificial Sequence
  Synthetic clockwise primer
ctagacgctgtagccttgat

SEQ ID NO: 12 - DNA, 20 bp, Artificial Sequence
  Synthetic counterclockwise primer
ctaaatcaacgcttagactt

SEQ ID NO: 13 - DNA, 27 bp, Artificial Sequence
  Synthetic forward primer
aaatccgcggttagtgtgtgtgtatta

SEQ ID NO: 14 - DNA, 28 bp, Artificial Sequence
  Synthetic reverse primer
aattgaattctaagcataatacatgact

SEQ ID NO: 15 - DNA, 28 bp, Artificial Sequence
  Synthetic forward primer
aattgaattcagacaataagaatgttct

SEQ ID NO: 16 - DNA, 28 bp, Artificial Sequence
  Synthetic reverse primer
aatagggccctgtctaaatgtgtattga

SEQ ID NO: 17 - DNA, 8 bp, Xenopus sp.
  misc_feature (1)..(8): FoxH1 consensus binding site
tgtnnatt

SEQ ID NO: 18 - DNA, 68 bp, Artificial Sequence
  Synthetic NRE sequence
ttagtgtgtgtgtattacctttaagacgtctgttttaaagtggcctgggcatcaatacacatttagac

SEQ ID NO: 19 - DNA, 36 bp, Drosophila melanogaster
attagtgtgtgtgtattagcatcaatacacatttag

SEQ ID NO: 20 - DNA, 18 bp, Tetraodon nigroviridis
tttctaaaatgttcatta

SEQ ID NO: 21 - DNA, 37 bp, Takifugu rubripes
ttaatagtaaaatacatcaatgataatcaacatttta

SEQ ID NO: 22 - DNA, 36 bp, Xenopus laevis
tttaaggtttgtgtattgagtctaatacacatagtc

SEQ ID NO: 23 - DNA, 36 bp, Mus musculus
cctgggcgcaatccacatgacgcaatccacatcttc